(19) United States
(12) Patent Application Publication
(10) Pub. No.: US 2008/0295847 A1
(43) Pub. Date: Dec. 4, 2008

(54) UNIQUE CONNECTORS FOR APPARATUS FOR VAP PREVENTATIVE VENTILATION OF INTUBATED CRITICALLY ILL PATIENTS

(75) Inventor: FRED G. GOBEL, Wilhemsfeld (DE)

Correspondence Address: DORITY & MANNING, P.A., POST OFFICE BOX 1449, GREENVILLE, SC, 29602-1449, US

(73) Assignee: KIMBERLY-CLARK WOLDWIDE, INC., Neenah, WI (US)

(21) Appl. No.: 11/755,349

(22) Filed: May 30, 2007

(51) Int. Cl. A61M 16/00

(52) U.S. Cl. 128/207.14

(57) ABSTRACT

An intermittent, large-volume flushing/suctioning scheme is provided. A specific and unique connector is used in conjunction with supplying a lavage solution to the patient via an intermittent, large-volume lavage/suctioning scheme that applies/removes the lavage solution via ports, which can be channels that are integrated into the shaft of a tracheal ventilation tube and/or an NG tube. The lavage solution is provided to the user in a specially designed container that allows for an exclusive fit between the container and the line that delivers lavage solution through the ports in the tracheal tube and the nasogastric tube. The connector element that connects to the container holding the lavage solution is specifically designed to prevent accidental connection to relevant supply lines or drainage lines that affect patient safety. A unique connector system is provided that prevents accidental mix-up of the line supplying the lavage solution to the patient with other patient supply and drainage lines in order to ensure safe application of the lavage solution for rinsing and subsequent suctioning thereof.

### CROSS-REFERENCE TO RELATED APPLICATIONS

N/A

### STATEMENT REGARDING FEDERALLY SPONSORED RESEARCH OR DEVELOPMENT

N/A

### BACKGROUND

The present disclosure relates to apparatus used to implement methods for reducing the incidence of ventilator associated pneumonia (VAP) in intubated and mechanically ventilated patients.

A mechanically ventilated patient typically would have a tube inserted into the trachea (tracheal tube or tracheostomy tube). The proximal end of the tube is connected to a ventilator to introduce air into the lungs and assist the patient's breathing. The distal end of the tube is disposed below the glottis and above where the trachea branches to the bronchial tubes. A cuff mechanically secures the distal end of the tube at this location within the trachea and permits positive ventilation pressures to build up in the lungs. The cuff typically is inflated and pressurized via a lumen formed in the wall of the tube. One end of the lumen is disposed outside the patient and connected to a source that maintains pressure in the cuff.

Mechanically ventilated patients are typically in a critically ill state and need to be fed with special solutions, which are administered through so-called naso-gastric (NG) tubes. Such NG tubes are inserted into the patient's esophagus with the distal end of the tube reaching into the patient's stomach. The NG tube also permits a continuous release of intra-gastric pressure that arises from the accumulation of gas and fluid inside the stomach. The naso-gastric tube can be held inside the lumen of the patient's esophagus by another cuff that typically is inflated and pressurized via a lumen formed in the wall of the NG tube.

Patients who are sufficiently ill to require mechanical ventilation via tracheal tubes and/or gastric feeding via NG tubes typically are fitted with other conduits such as intravenous lines and the like. Moreover, such patients require the care of many different persons during the course of the day. Such patients are not conscious of the attendance of hospital care givers or of the various medical treatments and procedures to which the patient is subjected.

VAP-preventive care measures that involve the application of various solutions, which may contain active agents or potentially toxic agents, are repeated in daily or shift intervals and thus present numerous opportunities for incorrectly administering such solutions. For example, the dispensers of such solution can be incorrectly attached to a lumen that is not meant to administer such solutions to the patient but rather is intended for the administration of different sorts of fluids to the patient or drainage of fluids from the patient. Such mistakes can have dire even fatal consequences for the patient, who frequently is not conscious or barely so during such administrations and thus incapable of protest. Moreover, even if conscious, the patient typically does not understand enough about what is required in order to correct any errors, even if the patient had the necessary level of perception and ability to communicate such errors to the attendant.

### SUMMARY

In accordance with the present disclosure, a specific connector is used in conjunction with supplying a lavage solution to the patient via an intermittent, large-volume lavage/suctioning scheme that applies/removes the lavage solution via ports, which can be channels that are integrated into the shaft of a tracheal ventilation tube and/or an NG tube. The overall procedure is described in commonly owned U.S. patent application Ser. No. 11/736,816, which is hereby incorporated herein in its entirety for all purposes.

The lavage solution desirably is provided to the user in a specially designed container that allows for an exclusive fit between the container and the line that delivers lavage solution through the ports in the tracheal tube and the nasogastric tube. The connecting element between the container holding the lavage solution and the catheter that delivers the lavage solution to the compartment formed in the patient is specifically designed to prevent accidental connection to relevant supply lines or drainage lines that affect patient safety. Desirably, a specific connector system is provided that prevents accidental mix-up of the line supplying the lavage solution to the stable space/compartment with other patient supply and drainage lines in order to ensure safe application of the lavage solution for rinsing and subsequent suctioning thereof.

Additional objects and advantages of the present disclosure will be set forth in part in the description that follows, and in part will be obvious from the description, or may be learned by practice of the present disclosure. The objects and advantages of the present disclosure may be realized and attained by means of the instrumentalities and combinations particularly pointed out in the appended claims.

The accompanying drawings, which are incorporated in and constitute a part of this specification, illustrate at least one presently preferred embodiment of the present disclosure as well as some alternative embodiments. These drawings, together with the description, serve to explain the principles of the present disclosure but by no means are intended to be exhaustive of all of the possible manifestations of the present disclosure.

### BRIEF DESCRIPTION OF THE DRAWINGS

A full and enabling disclosure of the present disclosure, including the best mode thereof to one skilled in the art, is set forth more particularly in the remainder of the specification, including reference to the accompanying figures, in which:

FIG. 1 includes a partial cross-sectional view of the anatomy of the human head, neck, stomach and upper thorax, schematically illustrates a container for lavage solution and a connector element between the container and the lavage port in accordance with the present disclosure.

FIGS. 2a, 2b, 2c and 2d schematically illustrate embodiments of connector systems in accordance with the present disclosure.

FIG. 2e is a top plan view taken from above the embodiment shown in FIG. 2c in the direction of the arrows 2e-2e therein.

FIG. 3a schematically illustrates a cross-sectional view of an alternative embodiment of connector systems in accordance with the present disclosure.

FIG. 3b is a cross-sectional view of components of the embodiment of FIG. 3a taken in the plane of the dashed line and looking in the direction of the arrows designated 3b-3b in FIG. 3a.

Repeat use of reference characters in the present specification and drawings is intended to represent the same or analogous features or elements of the present disclosure.

### DETAILED DESCRIPTION OF ILLUSTRATIVE EMBODIMENTS

Reference now will be made in detail to the presently preferred embodiments of the present disclosure, one or more examples of which are illustrated in the accompanying drawings. Each example is provided by way of explanation of the present disclosure, which is not restricted to the specifics of the examples. In fact, it will be apparent to those skilled in the art that various modifications and variations can be made in the present disclosure without departing from the scope or spirit of the present disclosure. For instance, features illustrated or described as part of one embodiment, can be used on another embodiment to yield a still further embodiment. Thus, it is intended that the present disclosure cover such modifications and variations as come within the scope of the appended claims and their equivalents.

The following acronyms or phrases will have the meanings ascribed to them hereafter. Enteral means relating to a method of nutrient delivery where fluid is given directly into the gastrointestinal tract. GI means gastro-intestinal. ICU means intensive care unit. NG means naso-gastric. The so called subglottic space is defined as the space that is created between the vocal cords and the upper (proximal) end of the cuff of an intubated, conventional, single-cuffed tracheal tube.

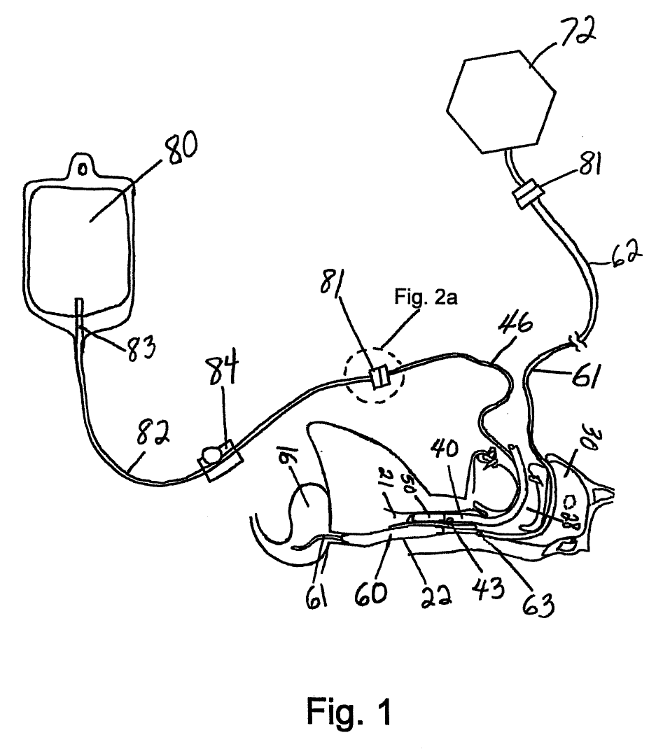

FIG. 1 illustrates the following portions of the anatomy relevant to the method herein. The trachea 21 forms an airway, and the esophagus 22 forms the digestive way leading to the stomach 16. The oral cavity is designated 28, and the nasal cavity is designated 30.

As noted above, the disclosure of commonly owned U.S. patent application Ser. No. 11/736,816 describes a method that includes the introduction of high-volume lavage solution into portions of the patient's subglottic, hypo-pharyngeal, oral and nasal, as well as pharyngeal and para-pharyngeal (sinusoidal) spaces. As schematically shown in FIG. 1 for example, the method involves connecting a supply container 80 of the lavage solution to a respective lumen 46 of a respective respiratory catheter device (tracheal tube) 40. A similar supply container (not shown in FIG. 1) of the lavage solution can be connected via a separate lumen 62 to a gastro-intestinal catheter device (NG tube) 61. Each of the supply containers 80, tracheal tube lumen 46 and NG tube lumen 62 is specifically designed to deploy the lavage solution into the patient. The unique connectors of the present disclosure are designed to securely prevent accidental connection of the lavage solution to other safety relevant patient catheters.

As schematically shown in FIG. 1 for example, a system for reducing the incidence of VAP in a mechanically ventilated, intubated patient, can comprise a first tube 40 having a proximal end and a distal end opposite said proximal end. The first tube 40 is configured for insertion into a patient's trachea 21 and can define a first port 43 disposed so as to be positioned proximal of a sealing cuff element 50 that is disposed inside the so-called subglottic space when the first tube 40 is inserted into the patient's trachea. A tracheal tube fitted with a so-called double cuff arrangement is referenced in U.S. Pat. No. 6,551,272, which is hereby incorporated herein in its entirety by this reference. As schematically shown in FIG. 1, the inclusion of a lavage/suction port 43 in the tracheal tube 40 provides the ability to introduce lavage solution through the port 43 into the patient and to remove the lavage solution from the patient by suctioning residual amounts of such solution through the port 43. A first lumen 46 can be disposed within the first tube 40 and have a distal end connected to the first port 43. A first cuff 50 can be configured surrounding the first tube 40 and disposed between the distal end of the first tube 40 and the first port 43. The first cuff 50 can be formed as a tamponade element 50 surrounding a distal portion of the trachea tube 40 and can be designed to fill out the total subglottic space. In the case of so-called subglottic tamponade cuffs, filling out the subglottic space and optionally exceeding the vocal chords into the supra-glottic section of the larynx, port 43 can be positioned proximally of the tampooning balloon 50, opening into the so-called hypo-pharynx.

As schematically shown in FIG. 1, a second tube 61 can be provided having a proximal end and a distal end opposite the proximal end. At least the distal end of the second tube 61 is configured for insertion into the patient's esophagus 22. The second tube 61 can define a second port 63 that is disposed so as to be positioned adjacent the patient's supra-glottic space when the second tube 61 is inserted into the patient's esophagus. A second lumen 62 can be disposed within the second tube 61 and have a distal end connected to the second port 63. A second cuff 60 can be configured surrounding the second tube 61 and disposed between the distal end of the second tube 61 and the second port 63. The second cuff 60 can be formed as a tamponade element 60 that surrounds a distal portion of the second tube 61 and can be disposed within the esophageal segment between the upper and lower esophageal sphincters. The second tube 61 can be provided by a suitable naso-gastric (NG) tube 61 with a sealing balloon element 60 such as is described in U.S. Pat. No. 6,551,272, which is hereby incorporated herein in its entirety by this reference for all purposes. As shown in FIG. 1 for example, the NG tube 61 equipped with an additional lavage/suctioning lumen 62 that opens into the hypo-pharyngeal space 23 via an opening 63 provides the ability to introduce lavage solution into the patient and to remove the lavage solution from the patient by suctioning residual amounts of such solution.

This naso-gastric tube 61 with sealing balloon element 60 is intended to minimize phyaryngeal directed ascension of gastric secretions and/or gastrically applied feeding solution in a ventilated patient. The sealing balloon element 60 also prevents the lavage solution from descending past the balloon 60 and entering the stomach 16. Thus, in accordance with an embodiment of the present disclosure, the first tube 40 and the second tube 61 and the first cuff 50 and the second cuff 60 are configured to cooperate so as to mechanically separate the patient's respiratory tract from communication with the patient's digestive tract in a manner that has the pharynx segmented off as a separated compartment that retains liquid.

A lavage supply container desirably is configured for selectively storing and supplying lavage solution and being selectively connected in communication with at least one of the first lumen 46 and the second lumen 62. FIG. 1 schematically illustrates a lavage container 80 that holds the supply of lavage solution that is introduced during the pharyngeal lavage step. Similar to intra-vascular infusion solutions, the lavage solution container 80 desirably is provided in the form of a soft-bag solution container 80. A regulating wheel 84 desirably can be provided for dosing of the flow of lavage solution.

In accordance with the present disclosure, the bag container 80 is directly connected with the lavage solution delivering tube element 82, thus preventing accidental connection to any other fluid delivering infusion set that might possibly be connected to the patient. As schematically shown in FIG. 1, the lavage solution container 80 desirably can be provided with an integrated tube 82 that desirably is fused or welded with bag 80 at site 83. With the injection hose element 83 integrated with the container 80 and with the container 80 lacking a puncture/injection port that typically is used with an infusion container, accidental usage of a standard infusion kit equipped with a Luer connector can be prevented.

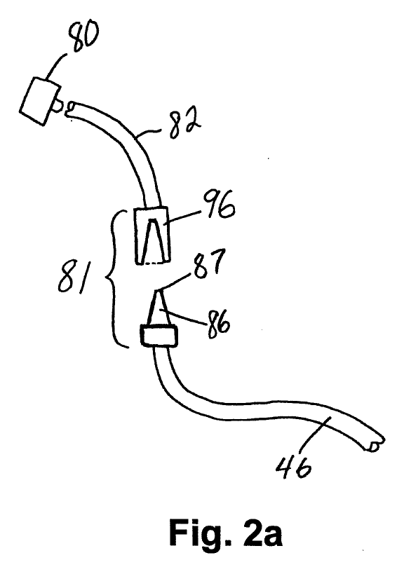

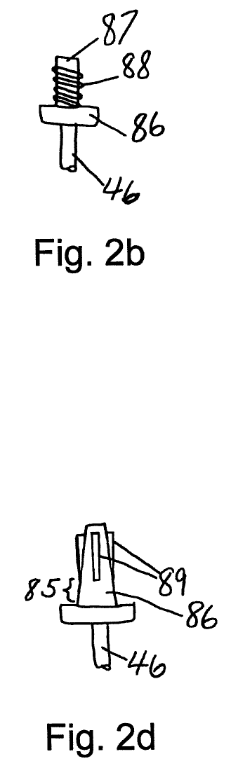

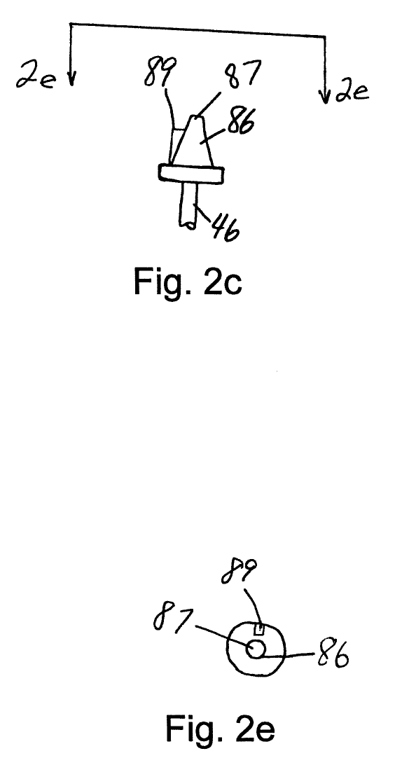

In accordance with the present disclosure, an exclusive fit between the lavage solution container 80 and the respective lavage ports 43, 63 of the solution supplying catheter (tracheal tube lumen 46, NG tube lumen 62) is critical for the safety of the pharyngeal care concept. Thus, a unique connector 81 that selectively and exclusively connects the lavage supply container 80 in communication with at least one of the first lumen 46 and the second lumen 62 is provided. As schematically shown in FIG. 1, a unique connector 81 in accordance with an embodiment of the present disclosure is disposed between the lavage solution container 80 and the lavage/suction port 43. In further accordance with the present disclosure and as schematically shown in FIG. 2a for example, the lavage solution container's tube element 82 desirably carries a specific connector 81 that is uniquely configured in a manner that prevents unintended connection to any intra-vascular lines, which typically carry Luer based connectors, further intra-thekal, para-spinal or epidural lines (usually Luer based), as well as connectors for gastro-intestinal and urinary tract catheters (typically equipped with funnel shaped connectors).

In accordance with the present disclosure, at least one of the first lumen 46 and the second lumen 62 terminates in a male connector piece 86 that forms a selectively detachable element of a unique connector element. FIG. 2a schematically illustrates an embodiment of a connector 81 that restricts connecting the lavage solution container 80 with any patient supply line other than the suctioning and lavage conduit 46 that has been integrated into the tracheal tube 40 that is used to intubate the patient. As schematically shown in FIG. 2a, the lavage conduit 46 is provided with a connector element 86 that desirably is configured as a male piece. Because typical patient supplying lines are provided with connector elements configured as female pieces, the connector element 86 of the lavage conduit 46 is configured as a male piece so as to eliminate the ability to attach the connector element 86 with any patient support devices that require the attachment of other typical patient supplying lines. The same arrangement applies equally well to the lavage/suctioning lumen 62 that has been integrated into the naso-gastric tube 61 as schematically shown in FIG. 1.

As schematically shown in FIG. 2a, the male connector piece 86 can be configured with a conical taper that is intentionally different than the taper of a typical Luer connector. As an alternative to a circular cross-section of the conus, an oval, triangular or other polygonal cross-section can be chosen. Desirably, the taper of the male connector piece 86 can be configured with a larger taper than the standard Luer taper so that connector piece 86 cannot be inserted into and connected by mistake into the opening of any supplying line that mates with a typical Luer male connector. The distal opening 87 of the connector piece 86 also desirably can be configured smaller than or larger than a standard Luer taper (or conus), so that a line equipped with a male Luer connector cannot be mistakenly substituted for connector piece 86. Accordingly, the distal end of the lavage solution delivering tube element 82 is provided with another unique connector element 96 that is configured as a female piece 96 that receives therein and connects to the corresponding male connector piece 86.

Alternatively, the thread mechanism can be integrated into a freely moving locking ring element, which can be located within the proximal segment of the connector and allow for free rotation of the locking ring element. This implementation is especially useful in case a direct counter directed twist movement of the sealing connector surfaces is not desired or not possible (e.g., conus with an oval cross-section or polygonal cross-section or with a keyed configuration), as this implementation permits the locking and securing ring element to be screwed onto the thread of the connecting counterpart.

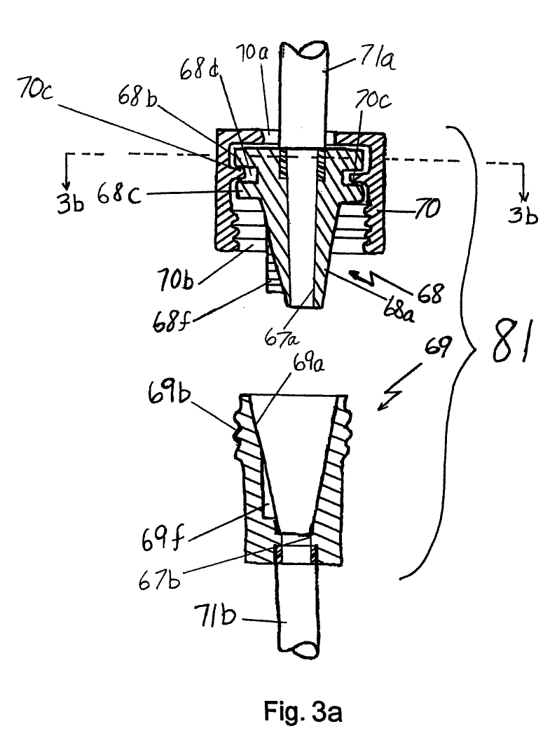

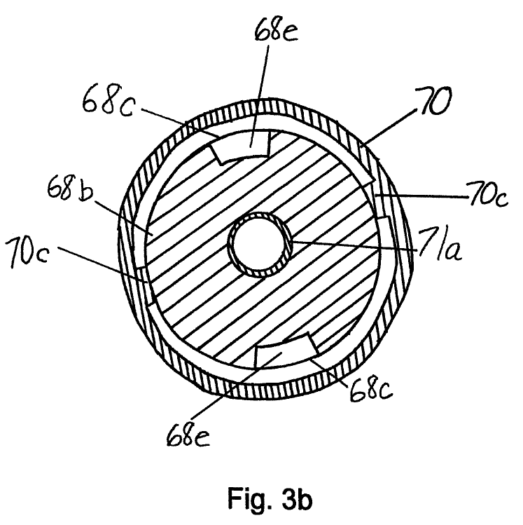

As schematically shown in FIG. 3a for example, an embodiment of the unique connector 81 can include a male connector piece 68 and a female connector piece 69 that can be secured to each other by a thread mechanism that allows the user to screw the elements 68, 69 together so as to press their sealing coni 68a, 69a into each other. A cup-shaped hood 70 can be rotatably connected to the male connector piece 68 and define a passage 70a through the proximal end of the hood 70. A lumen 71a that connects to the internal conduit 67a that is defined through the conus 68a can pass through the passage 70a defined in the proximal end of the hood 70. The distal end of the hood 70 can define an opening through which the male conus 68a can extend, and the interior cylindrical surface of the distal end of the hood 70 can define a screw thread 70b. The exterior surface of the mating female connector piece 69 can define a screw thread 69b that is configured to rotatably thread onto the screw thread 70b of the hood 70. A lumen 71b such as a supply line or a suction line can be connected to the internal conduit that is defined through the conus 69a of the female piece 69.

As schematically shown in FIG. 3a for example, the proximal end of the male connector piece 68 can define a pair of spaced apart radially extending flanges 68b, 68c that define therebetween an annular groove 68d. As schematically shown in FIG. 3b for example, two diametrically opposed circumferential gaps 68e are defined in the outermost rim of the proximal radially extending flange 68b. As schematically shown in FIG. 3a for example, located between the proximal end of the hood 70 and the distal end of the hood 70 that defines the screw thread 70a, the interior surface of the hood 70 can define a pair of opposed ribs 70c. The ribs 70c can be configured and disposed so that when the proximal end of the male connector piece 68 is moved axially into the interior of the hood 70, the ribs 70c can be oriented so as to pass through the pair of opposed circumferential gaps 68e that are defined in the proximal radially extending flange 68b.

As schematically shown in FIG. 3a for example, the exterior surface of the male conus 68a can be provided with a key 68f that is configured to be received axially in a slot 69f that is defined in the interior surface of the female conus 69a. The configuration of the key 68f and slot 69f prevents the male conus 68a from rotating with respect to the female conus 69a when the key 68f is fully inserted into the slot 69f.

As schematically shown in FIG. 1, the regulating wheel 84 desirably is disposed between the lavage solution container 80 and the site of the unique connector 81.

The fit of the male and female connector piece can be optimized or secured by a thread mechanism, which by screwing the pieces together, presses the sealing coni into each other. The thread can be designed in a way that the entire connector piece is twisted (the thread in that case would be integrated into the outer profile of the proximal connector end) similar to a so-called Luer lock mechanism.

As schematically shown in FIG. 2b, an alternative embodiment of the male connector 86 can be equipped with a thread 88 on its connecting surface that is configured so as to prevent accidental fit on a standard female piece of a Luer lock connector, thereby also preventing fit for example on funnel-shaped or conical-shaped connector pieces. The distal end of the lavage solution delivering tube element 82 can be provided with another unique connector element that is configured as a female piece that receives therein and connects to the corresponding male connector element 86, which is schematically shown in FIG. 2b.

As schematically shown in FIGS. 2c and 2d, further alternative embodiments of the male connector 86 can be equipped with axially directed bodies or bars 89 that are configured in order to prevent accidental fit onto a funnel shaped mating female connector. The distal end of the lavage solution delivering tube element 82 can be provided with another unique connector element that is configured as a female piece that can be provided with slots that receive the bars 89 therein and thereby can be connected to the corresponding male connector element 86, which is schematically shown in FIG. 2c or 2d. These bar elements 89 can span over the entire axially extending length of the taper (or conus) or can be positioned within the lower segment (FIG. 2c) or the upper segment (FIG. 2d) of the taper portion of the male connector 86. As schematically shown in FIG. 2d, disposing the axially directed bodies or bars 89 at the upper segment of the taper of the male connector 86, frees the lower part 85 of the taper portion of the male connector 86 to secure a tight fit over the remaining taper (or conus) section.

In accordance with an alternative embodiment of the present disclosure, a hand-held oral suctioning unit desirably can be connected directly onto the connector element of the lumen 46 that communicates with the lavage port 43 of the tracheal tube 40 in order to remove residual lavage solution from the subglottic space or the hypopharyngeal space. Similarly, in order to remove residual lavage solution from the subglottic space or the hypopharyngeal space, as shown schematically in FIG. 1 for example, a high volume suctioning device 72 desirably can be connected via the connector 81 directly to the lumen 62 that communicates with the port 63 of the NG tube 61. The high volume suctioning device 72 also can be connected to the lumen 46 that communicates with the flushing port 43 of the tracheal tube 40. The high volume suctioning device 72 desirably can be configured with its own disposable reservoir for receiving the residual fluids that are suctioned from these spaces and easily disposed of same.

In accordance with an alternative embodiment of the present disclosure, a space filling gel seal can be introduced from a container via a supply line that is provided with a uniquely configured connector 81 that cannot be connected with conventional patient supply lines. The gel can be so disposed on a portion of the exterior of the trachea tube 40 and contacting the first cuff 50 through port 43 via first lumen 46 for example. Similarly, the gel can be so disposed on a portion of the exterior of the NG tube 61 and contacting the second cuff 60 through port 63 via second lumen 62 for example.

While at least one presently preferred embodiment of the present disclosure has been described using specific terms, such description is for illustrative purposes only, and it is to be understood that changes and variations may be made without departing from the spirit or scope of the following claims.

1. A system for reducing the incidence of VAP in a mechanically ventilated, intubated patient, comprising:
a first tube having a proximal end and a distal end opposite said proximal end, said first tube being configured for insertion into a patient's trachea and defining a first port disposed so as to be positioned adjacent the patient's hypo-pharynx when inserted into the patient's trachea;
a first lumen disposed within said first tube and having a distal end connected to said first port;
a first cuff surrounding said first tube and disposed between the distal end of said first tube and said first port;
a second tube having a proximal end and a distal end opposite said proximal end, at least said distal end of said second tube being configured for insertion into the patient's esophagus, said second tube further defining a second port disposed so as to be positioned adjacent the patient's supra-glottic space when said second tube is inserted into the patient's esophagus;
a second lumen disposed within said second tube and having a distal end connected to said second port;
wherein said first and second tubes and said first and second cuffs are configured to cooperate so as to mechanically separate the patient's respiratory tract from communication with the patient's digestive tract in a manner that has the pharynx segmented off as a separated compartment that retains liquid;
a lavage supply container configured for selectively storing and supplying lavage solution, said container being selectively connected in communication with at least one of said first lumen and said second lumen;
a connector that selectively connects said lavage supply container in communication with at least one of said first lumen and said second lumen, said connector including a connector element; and
at least one of said first lumen and said second lumen terminates in a male connector piece forming a selectively detachable member of said connector element.

2. A system as in claim 1, wherein:
said male connector piece is configured with a conical taper that is configured sufficiently differently than the taper of a Luer connector such that said male connector piece cannot be connected to a Luer connector.

3. A system as in claim 2, wherein:
said male connector piece is configured with a conical taper that is a larger taper than the standard Luer taper.

4. A system as in claim 3, wherein:
said male connector piece is configured with a distal opening that is a larger opening than a standard Luer conus so that a line equipped with a male Luer connector cannot be mistakenly substituted for said male connector piece.

5. A system as in claim 1, wherein:
said male connector piece is configured with a taper that has an oval cross-section sufficiently differently than the taper of a Luer connector such that said male connector piece cannot be connected to a Luer connector.

6. A system as in claim 1, wherein:
said male connector piece is configured with a taper that has a polygonal cross-section sufficiently differently than the taper of a Luer connector such that said male connector piece cannot be connected to a Luer connector.

7. A system as in claim 1, wherein:
said male connector piece is configured with a thread that is configured so as to prevent accidental fit on a standard female piece of a standard Luer lock connector.

8. A system as in claim 1, wherein:
said male connector piece is configured with at least one axially directed body that is configured in order to prevent accidental fit onto a funnel shaped mating female connector piece.

9. A system as in claim 8, wherein:
said at least one axially directed body of said male connector piece is confined to the upper segment of the taper of the male connector piece.

10. A system as in claim 8, wherein:
more than one axially directed body of said male connector piece is confined to the upper segment of the taper of the male connector piece.

11. A system as in claim 1, further comprising:
a hood rotatably connected to said male connector piece and including a distal end that defines a screw thread.

12. A system as in claim 1, further comprising:
a suctioning device configured to remove from the intubated patient, dislodged and washed out residual colonized material, said suctioning device being selectively connected via said connector element to at least one of said first lumen and said second lumen.

13. A system as in claim 1, wherein:
said lavage supply container is configured as a soft bag with an integrated tube that has a proximal end fused or welded with the bag, and said integrated tube defines a distal end with a female connector piece that is configured to be connected with the male connector piece.

14. A system as in claim 1, wherein:
the lavage supply container lacks a puncture/injection port that typically is used with an infusion container.

15. A method for reducing the incidence of VAP in mechanically ventilated, intubated patients maintained in the supine position, comprising:
using a trachea tube surrounding by an inflatable cuff to separate the respiratory tract from communication with the digestive tract in a manner that has the pharynx segmented off as a separated compartment that retains at least a 10 cm column of liquid above the subglottic space in the patient;
connecting a container of lavage solution via a supply line to a lumen integrated with the trachea tube wherein the lumen is provided with a uniquely configured connector that cannot be connected with conventional patient supply lines; and
introducing lavage solution from the container via the lumen connected to a port defined in the trachea tube.

16. A method as in claim 15, further comprising:
using a suctioning device with a line that is provided with a uniquely configured connector that cannot be connected with conventional patient supply lines, to remove lavage solution from the intubated patient together with dislodged and washed out residual colonized material.

17. A method as in claim 16, wherein:
lavage solution is removed to a receptacle via a supply line that is provided with a uniquely configured connector that cannot be connected with conventional patient supply lines.

18. A method as in claim 15 further comprising:
introducing from a container via a supply line that is provided with a uniquely configured connector that cannot be connected with conventional patient supply lines, a space filling gel seal disposed on a portion of the exterior of said tracheal tube and contacting said cuff.
